(19) United States
(12) Patent Application Publication
(10) Pub. No.: US 2015/0306329 A1
(43) Pub. Date: Oct. 29, 2015

(54) PRESSURE OR FLOW LIMITING ADAPTOR

(71) Applicant: Cook Medical Technologies LLC, Bloomington, IN (US)

(72) Inventors: Elizabeth M. Brown, Bloomington (UD); Jeffry S. Melsheimer, Springville, IN (US)

(21) Appl. No.: 14/698,397

(22) Filed: Apr. 28, 2015

Related U.S. Application Data
(60) Provisional application No. 61/985,092, filed on Apr. 28, 2014.

(51) Int. Cl. A61M 16/04; A61M 16/20; A61M 16/08

(52) U.S. Cl. CPC A61M 16/0488; A61M 16/0434; A61M 16/0816; A61M 16/201; A61M 2202/0208; A61M 2205/3334

(57) ABSTRACT

An adapter for use in airway exchange procedure capable of being rapidly coupled and uncoupled to the catheter and being capable of regulating the flow of fluid delivered to the trachea of the patient. Oxygenation is received at the proximal end of the adapter and delivered to a catheter at the distal end. The adapter may include a relief valve to release excess fluid from the provided oxygenation. The adapter may also include a throttling valve to limit the flow of fluid within the valve, preventing excessive pressure or flow rate of breathable fluid to the catheter. During the procedure, the adapter and oxygenation source may be rapidly uncoupled and coupled to the catheter by a connector on the distal end of the adapter.

This application is a continuation-in-part of U.S. Provisional Application No. 61/985,092, filed Apr. 28, 2014, which is hereby incorporated by reference herein.

### BACKGROUND

The present disclosure relates generally to airway management devices. More particularly, the disclosure relates to an airway management device for use when oxygenating a patient during endotracheal tube intubation and/or extubation.

Airway exchange catheters are often used to oxygenate a patient during endotracheal tube (ETT) exchange. Removal of an endotracheal tube from the trachea of a patient is commonly referred to as extubation. Insertion of an endotracheal tube is commonly referred to as intubation. After an ETT has been positioned in the trachea of the patient for a period of time, a physician may determine that the existing ETT should be removed and exchanged for a new ETT, or in some instances, cleaned and repositioned in the trachea. The necessity to remove an existing ETT from the trachea of a patient and replace it with a new, or a cleaned, ETT may arise from, among other things, the physician's desire to utilize an ETT of a different size, the displacement of the existing ETT, or the malfunction of the existing ETT resulting from conditions such as blockage, e.g., as may be caused by patient mucous.

Proper placement and use of an airway exchange catheter (AEC) during endotracheal tube replacement is well known in the art. One particularly well-known method for replacing an ETT while maintaining oxygenation of the patient via an airway exchange catheter utilizes an adapter apparatus. The existing ETT is disconnected from a ventilator, and the airway exchange catheter is connected to the ventilator by way of a removable adapter, or connector, at the proximal end of the AEC. The AEC is then inserted into the lumen of the placed endotracheal tube. The adapter is configured to allow rapid connection, and disconnection, between the AEC and the ventilator. The AEC may be disconnected from the ventilator via the removable adapter as the ETT is removed from about the catheter. A replacement ETT may then be inserted over the AEC, whereupon the AEC is reconnected to the ventilator utilizing the removable connector. Once the replacement ETT is determined to be properly positioned in the trachea, the AEC is disconnected from the ventilator and removed from the interior space of the ETT. The ventilator is then connected to the replacement ETT. The AEC may have a distal portion of a lesser rigidity than the proximal portion of the catheter. By providing a catheter having a more flexible distal portion, the likelihood of irritating sensitive tracheal tissue is reduced when compared to a catheter having a more rigid distal portion.

When oxygenating a patient utilizing an AEC, the oxygen may be supplied by either of two general methods. One method is commonly referred to as low pressure oxygen insufflation. In this method, the adapter is provided with a conventional 15 mm ventilator fitting portion at its proximal end for connection to a mating fitting of a mechanical ventilation apparatus in well-known fashion. The other method is commonly referred to as high pressure, or “jet” ventilation. In this method, a luer lock connector is provided at the proximal end of the adapter instead of the 15 mm ventilator fitting portion. The luer lock connector is sized for connection to a mating connector on an auxiliary device, such as a jet ventilator. “Jet” ventilation is useful for short periods of time for patients who are unable to maintain sufficient oxygenation levels through natural ventilation.

For optimal results during oxygenation of a patient via jet ventilation, it is desirable to maintain oxygen flow within a generally controlled flow range, with a standard flow rate of about 15 L/minute. Those skilled in the art will appreciate that the desired range for a particular patient may vary based upon factors such as size and medical condition of the patient, the dimensions of the ETT and AEC, etc. For optimal results during oxygenation of a patient via jet ventilation, it is desirable to maintain oxygen pressure of 20-50 psi.

With existing AEC devices and adapters, it is generally necessary for the clinician to manually monitor the amount of oxygen administered to the patient, as well as the pressure of the oxygen flow. Additionally, high flow rates or high pressure from jet ventilation can cause barotrauma or volutrauma, severely damaging a patient's lungs. In order to minimize a possibility of undesired variations in such flow and/or pressure, it would be desirable to provide an adapter for an AEC that is capable of limiting or controlling air flow rate or pressure.

### SUMMARY

The present invention addresses the shortcomings of the prior art. In one form thereof, the invention comprises an airway management apparatus for engagement with a catheter for oxygenation of a patient. The airway management apparatus includes an endotracheal tube with a catheter inserted into the endotracheal tube. The endotracheal tube may be removed over the catheter so that it can be cleaned or replaced. During this time, the distal end of the catheter rests in the patient's trachea to ensure that the trachea remains open to the flow of oxygen and a pathway through the vocal chords is maintained.

Jet ventilation may be provided to the patient through the catheter. An adapter is coupled to the proximal end of the catheter while a source of jet ventilation is coupled to the adapter. The adapter has a valve to regulate the fluid flow of the ventilation to the patient. This valve may take the form of a relief valve which bleeds off excess fluid to prevent the flow of air to the patient above a maximum pressure or flow rate. Alternatively, the valve may take the form of a throttling valve which can be adjusted to permit a set flow or pressure of fluid which passes to the patient's trachea. The adapter may even have both a relief valve and a throttling valve to better regulate the fluid flow to the patient.

The adapter is designed to be easily and quickly coupled and uncoupled from the catheter. If the endotracheal tube is to be replaced, the adapter may be coupled and uncoupled to the catheter multiple times during a procedure to ensure that the patient is sufficiently oxygenated. To accomplish quick and easy coupling to the catheter, the adapter may have a series of compressible members configured to engage the proximal end of the catheter when a movable collar is pushed over them. When the collar retracts, the compressible members release, allowing the catheter to be uncoupled. Once the endotracheal tube is replaced into the trachea over the catheter, the ventilation source, adapter, and catheter may be removed.

### BRIEF DESCRIPTION OF THE DRAWINGS

FIG. 1 illustrates use of a prior art airway management apparatus during exchange of an endotracheal tube in a patient.

FIG. 2 is a cross-sectional view of the airway management adapter including a relief valve.

FIG. 3 is a cross-sectional view of the airway management adapter including a throttling valve.

FIG. 4 is a cross-sectional view of the airway management adapter including an alternative embodiment of the relief valve.

FIG. 5 is a cross-sectional view of the device showing both a relief valve and a throttling valve.

FIG. 6 is a cross sectional view of the device showing a relief valve with a wire guide port.

### DETAILED DESCRIPTION

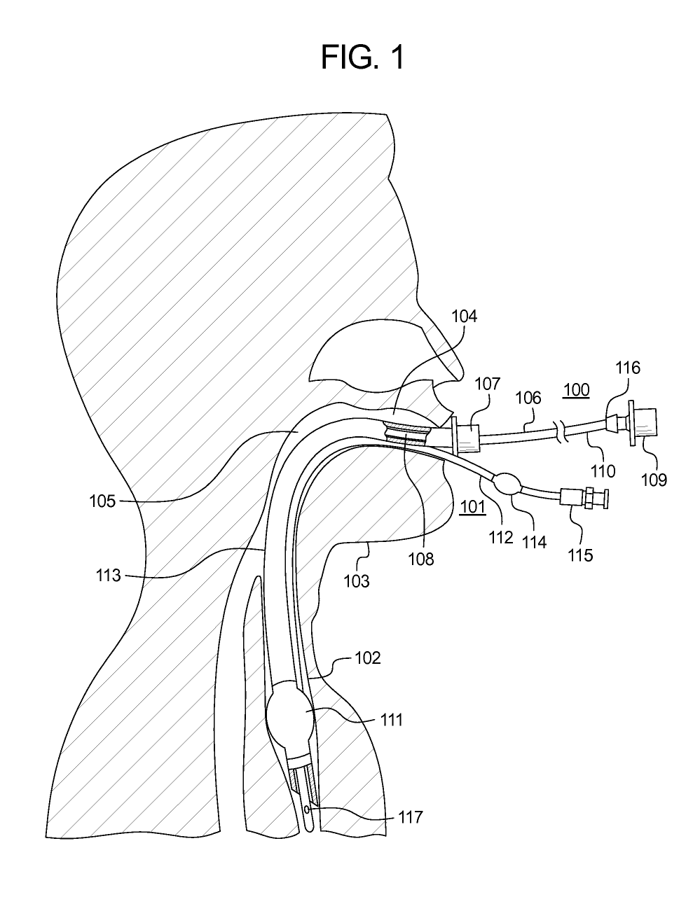

Referring now to the drawings, and particularly to FIG. 1, a well-known example of an airway management system is shown. In this embodiment, an endotracheal tube 113 is shown placed in the trachea 102 of a patient 103. As shown, an endotracheal tube 113 is commonly placed through the mouth 104 extending through an airway 105 into the trachea 102 of the patient. The endotracheal tube 113 is hollow and sufficiently rigid to maintain its tubular structure and permit the free flow of breathable fluid through a ventilating passageway 108 within the tube. Additionally, the endotracheal tube 113 may be sufficiently flexible to accommodate the curvature needed for its distal end to reach the trachea 102 while having a proximal end still extending from the mouth 104. Alternatively, the endotracheal tube 113 may be rigid with a preformed curve to accommodate the airway 105 of the patient 103. At the proximal end of the endotracheal tube 113, a ventilator connector 107 may be attached, which can be configured to connect to a ventilator to provide natural ventilation or jet ventilation to the patient 103 through the endotracheal tube 113.

In the particular embodiment shown in FIG. 1, an inflatable cuff 111 is also shown, surrounding the endotracheal tube 113 and attached near the distal end of the endotracheal tube 113. When inflated, the inflatable cuff 111 presses against the trachea 102 of the patient 103 ensuring that endotracheal tube 113 remains in a fixed position, while also preventing the endotracheal tube 113 from scratching against the trachea 102. The inflatable cuff 111 is inflated by delivery of a fluid through an inflation tube 112, which has a distal end attached to the inflatable cuff 111, and a proximal end which extends out of the mouth 104 of the patient 103. An inflatable balloon 114 may comprise a portion of the inflation tube 112 to provide a visual indication of the inflation of the cuff 111. Additionally, an airtight connector 115 may be coupled to the proximal end of the inflation tube 112 to ensure that the inflatable cuff 111 is sealed after it has been inflated.

Also shown in FIG. 1, a catheter 106 is shown within the ventilating passageway 108 of the endotracheal tube 113, with the catheter's 106 distal end extending into the trachea 102 of the patient 103. Similar to the endotracheal tube 113, the catheter 106 is tubular with a proximal 116 and distal opening 117 creating another ventilating passage. The catheter 106 must be sufficiently rigid to maintain its tubular structure and permit the free flow of fluids such as oxygen. The catheter 106 must also be sufficiently flexible to conform to the curve of the endotracheal tube 113 from the mouth 104 to the trachea 102 without kinking.

On the proximal end 110 of the catheter 106 an adapter 109 may be attached. This adapter 109 may be configured to receive a source of jet ventilation or may be unattached to a ventilator to provide natural ventilation. The adapter 109 is sized and configured so that the distal end of the adapter 109 has a first diameter which may attach to the proximal end 110 of the catheter 106, while the proximal end of the adapter 109 has a second, possibly distinct diameter, which may receive a ventilation tube 217. It may be advantageous to size the proximal end of the adapter 109 so that it has the same diameter as the ventilator connector 107 of the endotracheal tube 113, so that the same ventilation tube 217 may be received by either fitting.

The steps involved in replacing an endotracheal tube 113 placed in a patient 103 as shown in FIG. 1 vary depending on the amount of oxygenation of patient requires. Initially, the ventilating passageway 108 of the endotracheal tube 113 is open and is not obstructed by the catheter 106. To begin the procedure, the ventilation source, if connected, is removed from the ventilator connector 107 of the endotracheal tube 113. The ventilation source may then be attached to the adapter 109 so that oxygenation may occur through the catheter 106. The catheter 106 is then positioned in the endotracheal tube 113 so that the distal opening 117 of the catheter 106 extends into the trachea 102 of the patient 103, while the adapter 109 connected to the proximal end 110 of the catheter 106 remains outside of the endotracheal tube 113. The inflatable cuff 111 may now be deflated, so that the endotracheal tube 113 can be withdrawn over the catheter 106. It may only be necessary to clean the endotracheal tube 113, in which case, it can be partially withdrawn over the catheter so that it can be cleaned and then repositioned in the trachea 102. If, however the endotracheal tube 113 is to be replaced, then the catheter 106 is uncoupled from the adapter 109 and the endotracheal tube 113 is removed entirely from the trachea 102 over the catheter 106. The catheter 106 may then be recoupled to the adapter 109 to provide oxygenation to the patient if needed.

If the endotracheal tube has been chronically placed in the patient's 103 trachea 102, it is common for the tissue of the patient's 103 airway 105 to become inflamed thereby encapsulating the catheter 106 when the endotracheal tube 113 is removed. This inflammation may result in making natural ventilation through the catheter 106 insufficient to adequately oxygenate the patient. In this case, positive “jet” ventilation of oxygen can be applied from the ventilation source coupled to the adapter. This jet ventilation can be induced as needed to maintain oxygenation levels in the patient during the course of the procedure.

After the endotracheal tube 113 has been fully removed, a new endotracheal tube 113 may be placed in the airway 105. The catheter 106 must be first disconnected from the adapter 109, and then the endotracheal tube 113 may be inserted into the airway 105 over the catheter 106. If oxygenation is needed, the catheter 106 may be recoupled to the adapter 109, while the cuff 111 of the endotracheal tube 113 is being inflated. Once the endotracheal tube 113 is in place, the catheter 106 may be removed through the ventilating passageway 108 of the endotracheal tube 113. The ventilation source may then be decoupled from the adapter 109 and, if needed, attached to the ventilator connector 107 of the endotracheal tube 113.

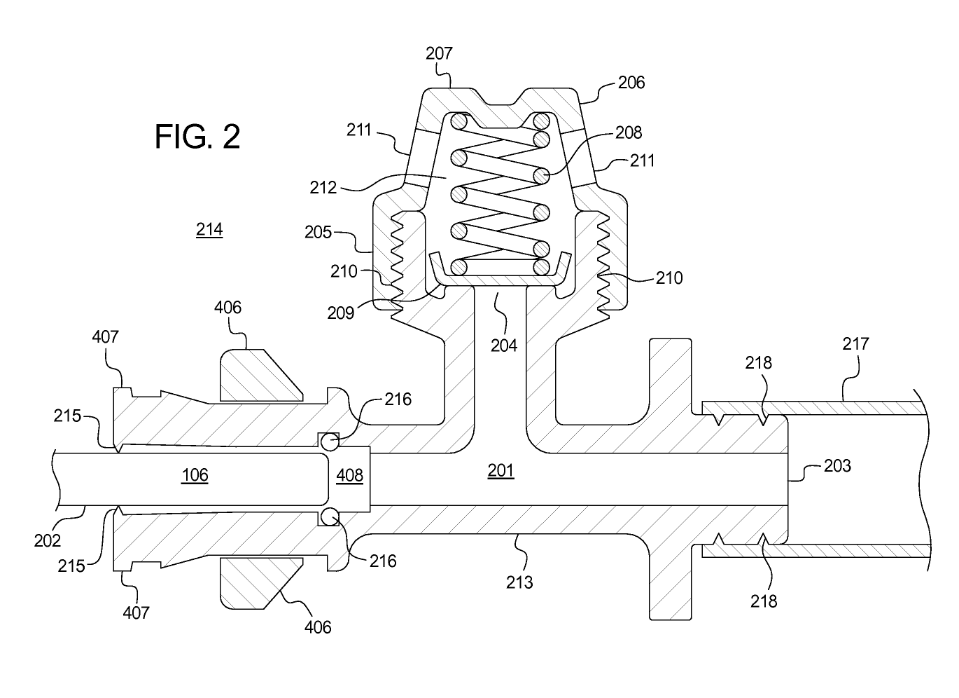

Referring to FIG. 2, a particular embodiment of the adapter 214 which engages with the catheter 106 is shown. The adapter 214 of FIG. 2 comprises a hollow main body 213 with three openings 202, 203, 204 through which a ventilation fluid, such as oxygen, air, or another breathable fluid, may flow. In this particular embodiment, the distal first opening 202 of the main body 213 of the adapter 214 engages with the catheter 106, while the proximal second opening 203 of the main body 213 is coupled to the ventilation tube 217. The third opening 204 of the main body 213 may include a valve 205 to regulate the flow of a breathable fluid through the adapter 109. In the embodiment shown in FIG. 2, the valve 205 is a relief valve 205 where a maximum pressure within the interior region 201 of the main body 213 causes the relief valve 205 to move from a first closed position to a second open position to permit a controlled flow of air through the third opening 204 and out of the interior region 201 of main body 213.

The embodiment of the relief valve 205 shown in FIG. 2 has a casing 206 which covers the third opening 204 of the main body 213 and forms a cavity 212 between the casing 206 and the third opening 204 of the main body 213. The casing 206 may be coupled to the hollow main body 213 through a variety of methods, however, FIG. 2 shows that the casing 206 and main body 213 are coupled by threads 210, where inward facing threads on the casing 206 may be screwed onto outward facing threads near the third opening 204 of the hollow main body 213. FIG. 2 also shows a seat 209 in the cavity 212 which rests on the third opening 204 of the main body 213, sealing it and preventing the flow of a fluid through the opening 204. This seat 209 is held on the opening 204 by a resistance mechanism 208 which applies a predetermined amount of force to the seat 204. The resistance mechanism 208 may take different forms, however. FIG. 2 shows a spring 208 extending from the casing 206 through the cavity 212 to the seat 209 as one possible embodiment of the resistance mechanism 208. FIG. 2 also shows that two vents 211 may be arranged on the outer surface 207 of the casing 206, which permit the flow of air from the cavity 212 to the atmosphere. There may be any number of vents 211 on the outer surface 207 of the casing 206, and the vents 211 may take many forms, such as being covered by a screen or shaped in such a way to direct the flow of fluid exiting the cavity 212.

The relief valve 205 shown in FIG. 2 remains closed as long as the pressure within the hollow main body 213 of the adapter 214 is below a maximum pressure. However, if the fluid pressure within the interior region 201 of the hollow main body 213 exceeds the maximum pressure, the fluid pressure will overcome the force of the resistance mechanism 208, and the seat 209 will lift, allowing the passage of the fluid from the third opening 204 into the cavity 212 of the valve 205 and through the vents 211 to the atmosphere. Once the fluid pressure in the interior region 201 of the hollow main body 213 drops below the maximum pressure, the force of the resistance mechanism 208 will force the seat 209 down, sealing the third opening 204.

In the embodiment shown in FIG. 2, the force applied to the seat 209 by the resistance mechanism 208 may be adjusted. Because the resistance mechanism 208 in FIG. 2 is a spring 208 which is placed with one end on the seat 209 and the other end on the casing 206, the resistance of the spring 208 can be adjusted by raising or lowering the height of the casing 206. The casing height can be adjusted by rotating the casing 206 about the threads 210 which couple the casing 206 to the adapter 214. In this way, the casing 206 shown in the embodiment of FIG. 2, functions as a control dial which can adjust the maximum pressure at which the valve 205 opens. When the casing height is raised, the resistance of the spring 208 is lessened and the maximum pressure for the valve 205 will decrease. When the casing height is lowered, the resistance of the spring 208 is increased and the maximum pressure for the relief valve 205 will increase. When the casing 206 is fully screwed onto the threads 210, the resistance mechanism 208 shown may produce a force which would require a maximum pressure within the hollow main body 213 to lift the seat 209 and permit the breathable fluid to escape from the adapter 109. The maximum pressure at which the seat 209 should be set to lift may vary depending on the physical characteristics of the patient, but typically, the seat 209 may be set to lift at a maximum pressure of approximately 50 psi.

Other equivalent alternative methods of adjusting the valve 205 may be employed to achieve the same effect, such as twisting a central shaft running through the casing 206 onto the seat 209. Furthermore, the threads 210 may have protrusions in the troughs of the threads 210 on either the adapter 214 or the casing 206 which would create a small amount of resistance to rotation. This feature would allow the user to rotate the casing 206 and click through a set of predetermined maximum pressures for the relief valve 205. This could be accomplished by adding a vertical groove interrupting the threads 210 on the adapter 214. For each full rotation of the casing 206, the protrusions would rest in the vertical groove, giving a small amount of resistance from being moved out of the groove. The threads 210 may also have a wall at the end of the trough of the threads 210 of either the casing 206 or the adapter 214 which would prevent the user from inadvertently separating the casing 206 from the adapter 214 by unscrewing it.

FIG. 2 also shows a connector on the distal end of the adapter 214. This connector comprises of a plurality of radially arranged, compressible members 407 and a movable collar 406 which encircles the distal end of the adapter 214. The compressible members 407 extend in a distal direction and are arranged to create a chamber 408 for receiving the proximal end 110 of a catheter 106. When the movable collar 406 is moved over the compressible members 407, the compressible members 407 are compressed around the outside of the catheter's 106 proximal end 110, holding the catheter 106 in place. Furthermore, the end of the compressible members 407 may comprise teeth 215 which focus the compressive force around the catheter 106, better holding it in place. Additionally, a seal 216 may be positioned in the proximal end of the chamber 408 to prevent fluid leakage while the catheter 106 is coupled to the adapter 214. When the collar 406 is moved off of the compressible members 407, the compressible members 407 are allowed to expand, releasing the catheter 106 from the adapter 214. The connector may be utilized to quickly apply and remove the adapter 109 from the catheter 106.

FIG. 2 also shows a threaded receptor on the proximal end of the adapter 214 for receiving a ventilation tube 217. The receptor may have a series of threads 218 on the outside of the receptor to more firmly receive a ventilation tube 217 having threads 218 on the inside of its distal opening. Threads 218 on the receptor and ventilation tube 217 serve to prevent leakage of breathable fluid passing from the ventilation tube 217 to the adapter 214. Additionally, a threaded connection fixedly couples the ventilation tube 217 to the adapter 214 and prevents the ventilation tube 217 from inadvertently separating during the procedure.

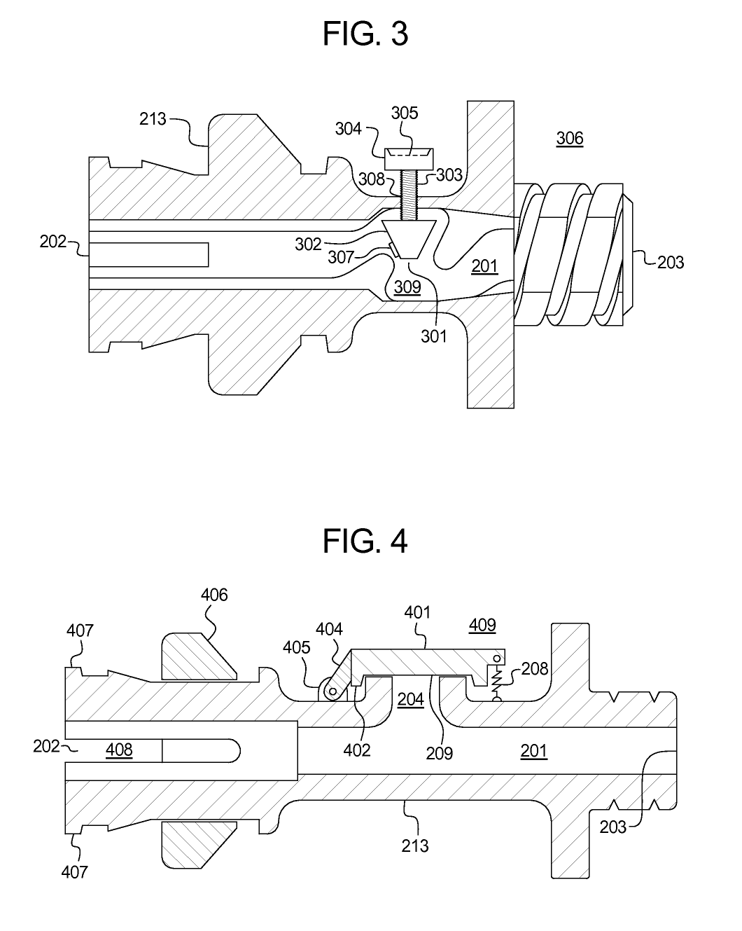

Referring to FIG. 3, a particular embodiment of the adapter 306 is shown wherein a throttling valve 309 is located in the interior region 201 of the hollow main body 213. The throttling valve 309 is positioned at some point between the distal and proximal ends of the adapter 306, and can be moved between an open position and an at least partially closed position to regulate the fluid flowing through the distal end of the hollow main body 213. In the particular embodiment shown in FIG. 2, the throttling valve 309 has an aperture 301 in the hollow main body 213 through which the fluid may flow. This aperture 301 may be defined by one or more elements located within the hollow main body 213. Additionally, the throttling valve has a seat 302 in the hollow main body 213 which may be placed in the aperture 301 by movement of a stem 303 coupled to the seat 302. The stem 303 extends through a sealed stem opening 308 in the hollow main body 213 where it is coupled to a valve control surface 304 which may be adjusted. A groove 305 may be provided on this control surface to facilitate easier manipulation of the throttling valve 309. The seat 302 shown in FIG. 2 may have a partial conical shape so that as it is extended into the aperture 301, it reduces the cross-sectional area of the aperture 301 through which the fluid may flow. Some embodiments may allow the seat 302 to fully occlude the aperture 301, but this is not required. Alternatively, to ensure that breathable fluid flow is not completely stopped by the adapter 306, it may be desirable to include a stop 307 or ridge on the seat 302 of the throttling valve 309 which, as the seat 302 is lowered, would contact the wall of the aperture 301 to prevent the throttling valve 309 from completely sealing. Alternatively, it may be desirable to not include a stop 307 and allow the stop 302 to seal the aperture 301. Reducing the cross-sectional area of the aperture 301 could have numerous effects depending upon the characteristics of the fluid flowing through the adapter 306. However, if the fluid is a gas, such as oxygen or air, reducing the cross-sectional area of the aperture 301 will cause a decreased flow rate and a predictable pressure decrease from the proximal end to the distal end of the adapter 306.

Referring to FIG. 4, an embodiment of the adapter 409 with an alternative design for a relief valve 205 is shown. In this embodiment, the casing and seat are integrated into a single cap 401. This cap 401 is arranged on the third opening 204 of the hollow main body 312 so that the seat 401 seals the third opening 204 and may include a lip 402 which encircles a portion of the third opening 204. This cap 401 is coupled to an arm 404 which rotates about a hinge 405. Force is applied to the cap 401 by a resistance mechanism 208 to ensure that the cap 401 is sealed to cover the third opening 204 of the hollow main body 213. In the embodiment shown, this resistance mechanism 208 takes the form of a spring 208 which extends from the cap 401 to an anchoring point on the hollow main body 213, but other variations may be used, such as a pin within a slot or by utilizing the weight of the cap 401. When the fluid pressure in the hollow main body 213 reaches a certain maximum pressure, the pressure force on the cap 401 overcomes the resistance mechanism 208 causing the cap 401 to lift. Excess fluid is then vented from the third opening 204 out the side of the cap 401. Once the pressure decreases below the maximum pressure, the cap 401 lowers, sealing the third opening 204 of the hollow main body 213.

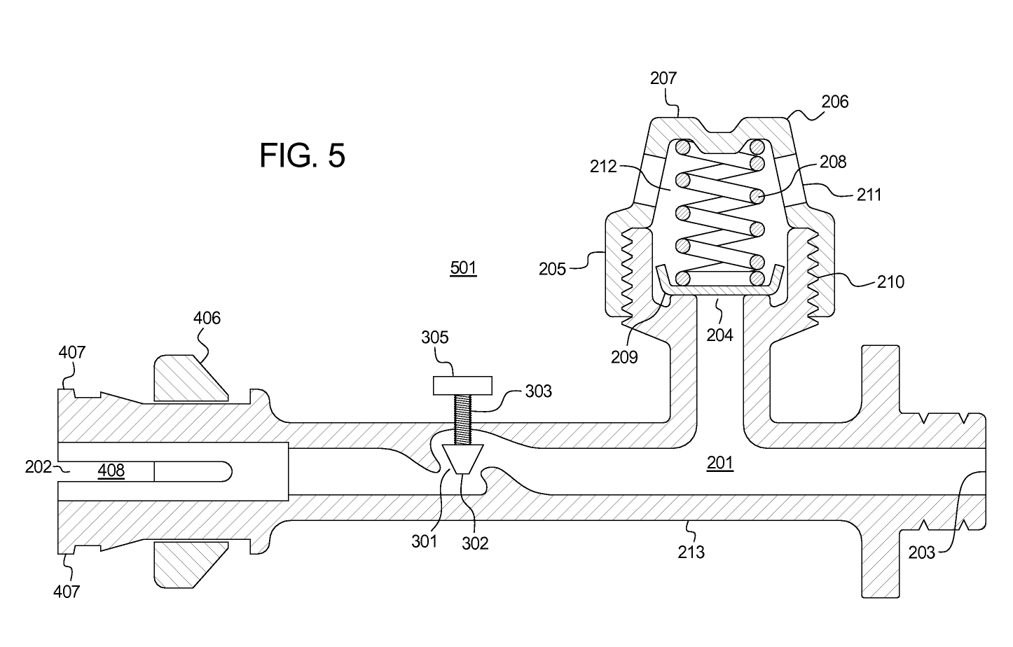

Referring to FIG. 5, it is possible and may be advantageous that the adapter 501 have both a throttling valve and a relief valve 205. In one possible embodiment, the throttling valve 309 would be placed closer to the distal end of the adapter 501, while the relief valve 205 would be placed closer to the proximal end. The restricted aperture 301 of the throttling valve 309 may create a higher back pressure on the proximal side of the aperture 301, depending upon the type of ventilation source connected to the proximal end of the adapter 501. In such a case, the relief valve 205, situated proximally to the throttling valve 309, would be configured to decrease this excess pressure on the proximal side of the adapter 501.

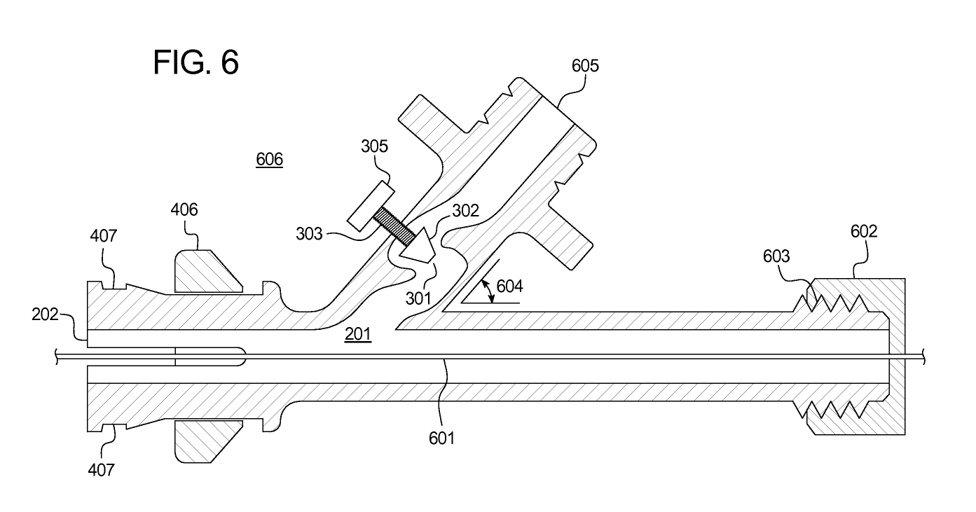

Referring to FIG. 6, an embodiment of the adapter 606 is shown incorporating a port 602 with a wire guide 601. In some cases, a wire guide 601 may be helpful during an airway exchange procedure to secure access to either the left or the right mainstream bronchus. To make positioning of the wire guide 601 less difficult, a wire guide port 602 is placed on the proximal end of the adapter 606 so as to provide a straight path for the wire guide 601 into the catheter 106. In such a situation, ventilation could be coupled to the adapter 606 by a branch at an angle 604 to the axis of the adapter 606 and through a different opening 605 in the adapter 109. If a throttling valve 309 is used in such an embodiment, it may be advantageous to place the throttling valve 309 in the angled branch so that it does not interfere with the path of the wire guide 601 from the proximal end to the distal end of the adapter 606.

Accordingly, it is now apparent that there are many advantages of the invention provided herein. In addition to the advantages that have been described, it is also possible that there are still other advantages that are not currently recognized but which may become apparent at a later time.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to embrace them.

We claim:

1. An airway management system for oxygenating a patient, comprising:
an endotracheal tube comprising a proximal end, a distal end, and a passageway extending therethrough;
a catheter comprising a proximal end and a distal end wherein the catheter is sized and configured to pass through the endotracheal tube;
an adapter comprising a hollow main body having a plurality of openings into an interior region thereof, wherein a first opening is arranged on the distal end of the hollow main body for coupling to said catheter, and a second opening is coupled to an oxygenation source; and
a valve coupled to the hollow main body of the adapter, wherein the valve is moveable from a first position to a second position to regulate a flow of a breathable fluid from the first opening to the second opening through the distal end of the hollow main body.

2. The airway management system of claim 1, wherein the valve comprises a relief valve arranged on a third opening of the hollow main body, and wherein a maximum pressure within the interior region of the hollow main body moves the relief valve from a first closed position to a second open position to limit the pressure of the pressure of the breathable fluid through the valve.

3. The airway management system of claim 2, wherein the relief valve comprises:
a casing comprising an outer surface and a cavity, wherein the casing is coupled to the hollow main body of the adapter;
a seat arranged within the cavity of the casing, said seat for sealing the third opening of the hollow main body;
a resistance mechanism coupled to the seat, wherein the resistance mechanism applies force to the seat for sealing the third opening of the hollow main body until the maximum pressure is reached in the interior region of the hollow main body; and
a vent arranged on the outer surface of the casing, for allowing passage of said fluid from the cavity of the casing to an atmosphere.

4. The airway management system of claim 2, further comprising a control dial coupled to the relief valve for adjusting the maximum pressure at which the valve opens to permit the flow of said fluid through the valve.

5. The airway management system of claim 2, further comprising a throttling valve arranged between the proximal end and the distal end, within the interior region of the hollow main body, wherein the throttling valve is moveable between respective partially closed and open positions to regulate said fluid flowing through the distal end of the hollow main body.

6. The airway management system of claim 1, wherein the valve comprises a throttling valve arranged between the proximal end and the distal end of the adapter, within the interior region of the hollow main body, wherein the throttling valve is moveable between an open position and an at least partially closed position to regulate said fluid flowing through the distal end of the hollow main body.

7. The airway management system of claim 6, wherein the throttling valve comprises:
an element within the hollow main body which defines an aperture through which said fluid may flow through the distal end of the hollow main body;
a seat positioned within the hollow main body which is configured to restrict at least some of the flow of said fluid through the aperture; and
a stem extending through a third opening in the hollow main body which is coupled to the seat, wherein the stem may be adjusted to move the seat to restrict the flow of said fluid more or less through the aperture.

8. The airway management system of claim 7, further comprising a stop arranged on the seat of the throttling valve, wherein the stop prevents the seat from sealing the aperture.

9. The airway management system of claim 1, wherein the oxygenation source comprises jet ventilation.

10. The airway management system of claim 1, wherein the adapter further comprises a connector for engagement with the proximal end of said catheter, said connector comprising a plurality of radially compressible members extending in a distal direction, said compressible members circumferentially aligned to define a chamber for receiving the proximal end of said catheter, said connector further comprising a movable collar positioned for selectively compressing a distal end portion of said compressible members around the proximal end of said catheter, and releasing said compressible members from around the proximal end of said catheter.

11. A method for oxygenating a patient during removal of an endotracheal tube, comprising:
positioning a catheter having a distal end and a proximal end wherein the distal end of the catheter extends through an endotracheal tube and into a trachea of the patient;
coupling an adapter to the proximal end of the catheter, wherein the adapter comprises a hollow main body having a plurality of openings into an interior region thereof, wherein a first opening is arranged on a distal end of the hollow main body and is reversibly coupled to the proximal end of the catheter, and a second opening is arranged on a proximal end of the hollow main body;
receiving an oxygenation source to a second opening on the hollow main body of the adapter; and
regulating air flowing through the interior region of the hollow main body by movement of a valve coupled to the hollow main body of the adapter.

12. The method of claim 11, further comprising:
at least partially withdrawing the endotracheal tube from the trachea over the catheter;
uncoupling the proximal end of the catheter from the first opening at the distal end of the hollow main body of the adapter;
removing the endotracheal tube over the catheter; and
after removing the endotracheal tube, re-coupling the proximal end of the catheter to the adapter to provide oxygenation as needed.

13. The method of claim 12, further comprising:
inserting a second endotracheal tube over the proximal end of the catheter, while the catheter and adapter are uncoupled, and advancing the proximal end of the second endotracheal tube into the trachea,
after inserting the second endotracheal tube, re-coupling the proximal end of the catheter to the adapter to provide oxygenation as needed; and
withdrawing the catheter from the trachea through the second endotracheal tube.

14. The method of claim 11, wherein the valve comprises a relief valve arranged on a third opening of the hollow main body, and wherein a maximum pressure within the interior region of the hollow main body moves the relief valve from a first closed position to a second open position to permit a controlled flow of a fluid through the valve.

15. The method of claim 14, wherein the relief valve comprises:
a casing comprising an outer surface and a cavity, wherein the casing is coupled to the hollow main body of the adapter;
a seat arranged within the cavity of the casing, said seat for sealing the third opening of the hollow main body;
a resistance mechanism coupled to the seat, wherein the resistance mechanism applies force to the seat for sealing the third opening of the hollow main body until a maximum pressure is reached in the interior region of the hollow main body; and
a vent arranged on the outer surface of the casing, for allowing passage of the fluid from the cavity of the casing to an atmosphere.

16. The method of claim 14, further comprising a control mechanism coupled to the relief valve for adjusting a maximum pressure at which the relief valve opens to permit the flow of the fluid through the relief valve.

17. The method of claim 11, wherein the valve comprises a throttling valve arranged between the proximal end and the distal end of the adapter, within the interior region of the hollow main body, wherein the throttling valve is moveable between an open position and an at least partially closed position to regulate a fluid flowing through the distal end of the hollow main body.

18. The method of claim 17, wherein the throttling valve comprises:
an element within the hollow main body which defines an aperture through which said fluid may flow through the distal end of the hollow main body;
a seat positioned within the hollow main body which is configured to restrict at least some of the flow of said fluid through the aperture; and
a stem extending through a third opening in the hollow main body which is coupled to the seat, wherein the stem may be adjusted to move the seat to restrict the flow of said fluid more or less through the aperture.

19. The method of claim 18, further comprising a stop arranged on the seat of the throttling valve, wherein the stop prevents the seat from sealing the aperture.

20. The method of claim 11, wherein the adapter further comprises a connector for engagement with the proximal end of said catheter, said connector comprising a plurality of radially compressible members extending in a distal direction, said compressible members circumferentially aligned to define a chamber for receiving the proximal end of said catheter, said connector further comprising a movable collar positioned for selectively compressing a distal end portion of said compressible members around the proximal end of said catheter, and releasing said compressible members from around the proximal end of said catheter.
